(19) United States
(12) Patent Application Publication
(10) Pub. No.: US 2011/0044917 A1
(43) Pub. Date: Feb. 24, 2011

(54) Dental composition for treating peri-implantitis

(76) Inventor: Alessandro Tosetti, Firenze (IT)

Correspondence Address: DENNISON, SCHULTZ & MACDONALD, 1727 KING STREET, SUITE 105, ALEXANDRIA, VA, 22314, US

(21) Appl. No.: 12/736,340

(22) PCT Filed: Mar. 27, 2009
(86) PCT No.: PCT/IB2009/005094

§ 371 Date: Oct. 28, 2010

(30) Foreign Application Priority Data
Mar. 31, 2008 (IT) PI2008A000025

(51) Int. Cl. A61K 31/496; A61P 1/02; A61P 29/00; A61P 31/00

(52) U.S. Cl. 424/54

(57) ABSTRACT

A composition for treating peri-implantitis, including bacterial inflammatory processes that occur in a bone tissue close to a dental implant, comprising at least one biocompatible acrylic resin which is impermeable to water, but insoluble to water; an antibiotic mixture; an organic solvent that is adapted to gradually evaporate, such that said at least one resin forms a protective film that gradually releases said antibiotic mixture. The antibiotic mixture comprises piperacillin sodium and tazobactam sodium in a predetermined weight ratio. In particular, said or each acrylic resin is permeable to water, but insoluble to water, ensuring the permanence of the film in the mouth even if saliva is present and at the same time releases piperacillin sodium and tazobactam sodium. The composition achieves a gradual release of the therapeutic agent directly close to the pathogenic process and allows elimination of bacterial deposits from the raw surface of the dental implant.

### DESCRIPTION

1. Field of the invention

The present invention relates to a composition for dental use, for treating oral pathologies, in particular, peri-implantitis, i.e. bacterial inflammatory processes that hinder osteointegration and that occur in a bone tissue close to a dental implant after its insertion.

2. Description of the Technical Problem

In particular, inflammatory processes that take place close to a dental implant may cause two different symptomatologies, which are respectively known as peri-implant mucositis and peri-implantitis. The former, which is defined as a reversible modification of peri-implant soft tissues without bone loss, is limited to the peri-implant soft tissue surface and involves a bacterial flora like that can be observed in periodontal diseases, even if normally with low subjective symptoms. The latter, instead, affects the deep soft tissues and the bone peri-implant, and it has been defined as an inflammatory process that affects the tissues that are close to an osteointegrated implant in such a way to cause a loss of alveolar support bone.

In the etiology of peri-implant tissue inflammations, an important role is played by bacteria, which grow on the surface of the implant. Some investigations have shown abundance of plasma cells and PMN (polymorphonuclear leukocytes), and have demonstrated that peri-implantitis is always associated with migrating, Gram-negative Anaerobic microorganisms, which may be facultative bacteria, such as Bacteroidaceae, Actinobacillus Actinomycetemcomitans and Spirochaetes. Therefore, the elimination of such bacteria is an indispensable step that must be carried out to resolve the pathology.

However, the elimination of such bacterial deposits on the implant surface is not easy. Normally, these deposits can be mechanically removed (debritment), even if, in addition, use of antimicrobial substances is advised in order to increase the effect of the debritment, in particular, antimicrobial aids are used that release several days high doses of antibiotics at the site that is affected by the pathology.

Therapeutic attempts for assisting osteointegration have been made with antibiotics or anti-inflammatory drugs, as well as possible curettage operations of the affected area. Furthermore, a sterilising therapy may be successfully used, by means of a laser, possibly in association with the use of Chlorhexidine.

Other treatments provide the use of specific instruments (non metallic curettes) that may be associated with administration of antiseptic products such as Chlorhexidine digluconate during 3 or 4 weeks, systemic antibiotic treatment with ornidazole (2×500 mg/die during ten days) or amoxicillin +clavulanic acid (2 g/die for ten days), topic antibiotic treatment with Slow Releasing Devices systems such as Hc tetracycline fibres or metronidazole gel. To treat the most serious cases, a regenerative technique may be used to recover the lost bone tissue by means of a membrane that may eventually be associated with grafts, until the fixture is explanted and then replaced immediately (wide diameter) or after a GBR therapy.

Peri-implantitis can also be treated by means of a systemic pharmacologic therapy based on ornidazole that is combined to a topic pharmacologic therapy with metronidazole gel, these therapies associated with the use of chlorhexidine and with an occlusal balancing of the implant, in such a way that a peri-implant regenerative therapy is carried out when an inflammation is not present.

However, the areas of the implant that is contiguous to the bone have often a raw surface, to assist implant adhesion and osteointegration, and can remain contaminated also after an antimicrobial treatment has been made, with subsequent further bone loss and formation of a peri-implant pocket.

Among the above-mentioned systems, however, there are no compositions that show a high effectiveness both in terms of antimicrobial agent slow release and of healing capacity with respect to a raw implant surface.

### SUMMARY OF THE INVENTION

It is therefore a feature of the present invention to provide a new composition for treating peri-implantitis that allows a gradual release of the therapeutic agent close to a pathogenic process.

It is another feature of the present invention to provide a new composition for treating peri-implantitis, which is suitable for eliminating the bacterial deposits from the raw surface of the dental implant.

These and other objects are achieved by a composition, according to the invention, for treating a peri-implantitis, i.e. a bacterial inflammatory process that occur in the bone tissue that is close to a dental implant, said composition comprising:

at least one biocompatible acrylic resin, said or each acrylic resin permeable to water, but insoluble in water;
an antibiotic mixture;
an organic solvent, in use, adapted to gradually evaporate, whereby said at least one resin forms a protective film that gradually releases said antibiotic mixture.
The main feature of the composition is that said antibiotic mixture comprises piperacillin sodium and tazobactam sodium according to a predetermined weight ratio.

In particular, said or each acrylic resin is permeable to water but insoluble in water, it ensures the permanence of the film in the mouth even if saliva is present, and it releases at the same time piperacillin sodium and tazobactam sodium.

In particular, said acrylic resin is such that said protective film has physical characteristics that remain substantially unchanged during 7 to 10 days, after which it deteriorates and disappears from the site where it has been applied.

Preferably, said or each biocompatible acrylic resin is selected from the group comprised of:

### EUDRAGIT RL;

### EUDRAGIT RS;

a mixture thereof.

The acrylic resins are pharmacologically inactive and have a high tolerability both at the skin level and at the mucous level, therefore mucous irritation and sensitisation is prevented.

Advantageously, the organic solvent is ethyl alcohol.

In particular, the weight ratio between tazobactam sodium and piperacillin sodium is set between ⅙ and 1/10, advantageously it is set between 1÷7 and 1÷9, preferably it is 1÷8. In particular, with a ratio of 1÷8 the best synergistic behaviour of the two antibiotics is obtained.

Furthermore, tazobactam sodium and piperacillin sodium are not orally bioavailable. Therefore, they give to the composition according to the invention optimal systemic tolerability features. These antibiotics, in fact, are not absorbed by the mucosa and therefore a therapeutically active plasma concentration is expected to be achieved.

Advantageously, the above described composition is associated with a means for administrating it to the peri-implant region close to a dental implant that is affected by the inflammatory process, in particular, the composition is associated with a dental kit which comprises a flexible blunt needle.

In particular, since the dental implant comprises a raw surface that is exposed to oral fluids, because a vertical bone loss has occurred and due to the local inflammatory process a peri-implant pocket has been created, the above described composition is applied in such pocket, in such a way that it adheres to the raw surface of the implant and to the mucous surface of the pocket same. Therefore, the solution can be advantageously applied into a peri-implant pocket by bending the blunt tip needle, miming a periodontal probe, positioning the tip of the blunt needle close to the base of the pocket and injecting the product until the solution reaches the upper edge of the gum. Then, after drawing the needle out of the pocket, an air jet is blown on the solution that has just been applied (during ca.10 sec.) to assist solvent evaporation and resin/s adhesion (in the form of a film) on the treated zone.

According to another aspect of the invention, a kit for dental use, in particular, for treating peri-implantitis comprises:

a first vial containing a hydroalcoholic solution comprising:
an organic solvent;
at least one biocompatible acrylic resin, said or each acrylic resin permeable to water, but insoluble in water;
   a second vial that contains an antibiotic powder mixture of antibiotics, said powder mixture comprising piperacillin sodium and tazobactam sodium in a determined weight ratio.

In particular, the weight ratio between tazobactam sodium and piperacillin sodium is set between 1 to 6 and 1 to 10.

Advantageously the weight ratio between tazobactam sodium and piperacillin sodium is set between 1 to 7 and 1 to 9.

Preferably, the weight ratio between tazobactam sodium and piperacillin sodium is 1 to 8.

In particular, the powder mixture of the second vial comprises:

100 mg of piperacillin sodium;
12,5 mg of tazobactam sodium.

Advantageously, the hydroalcoholic solution has the following composition:

EUDRAGIT RS: between 5% and 10% by weight;
EUDRAGIT RL: between 4% and 6% by weight;
deionised water: between 10% and 20% by weight;
ethyl alcohol: between 70% and 75% by weight.

Advantageously, the first vial contains 1 ml of hydroalcoholic solution.

Advantageously, the dental kit comprises, furthermore, at least one syringe equipped with a disposable perforating needle that is used for drawing the hydroalcoholic solution from the first vial and for putting it into the second vial.

The kit can, furthermore, comprise an injection needle by means of which the composition, according to the invention, is applied into the region that is affected by the inflammation, i.e. into a peri-implant pocket. More in detail, the injection needle may have a blunt tip similar to a periodontal probe.

The following are examples of possible compositions for dental use for treatment of the peri-implantitis, according to the present invention. Such examples are therefore not limitative for the present invention.

### EXAMPLE 1

Eudragit RS: 7% (w/w)
Eudragit RL: 5% (w/w)
Deionised water: 15% (w/w)
Ethyl alcohol: 73% (w/w).

### EXAMPLE 2

Eudragit RS: 10% (w/w)
Eudragit RL: 4% (w/w)
Deionised water: 15% (w/w)
Ethyl alcohol: 71% (w/w).

### EXAMPLE 3

Eudragit RS: 10% (w/w)
Eudragit RL: 6% (w/w)
Deionised water: 16% (w/w)
Ethyl alcohol: 70% (w/w).

### EXAMPLE 4

Eudragit RS: 5% (w/w)
Eudragit RL: 4% (w/w)
Deionised water: 16% (w/w)
Ethyl alcohol: 75% (w/w).

### EXAMPLE 5

Eudragit RS: 5% (w/w)
Eudragit RL: 6% (w/w)
Deionised water: 19% (w/w)
Ethyl alcohol: 70% (w/w).

1. A composition for treating a peri-implantitis, said composition comprising:
at least one biocompatible acrylic resin, said or each acrylic resin permeable to water, but insoluble in water;
an antibiotic mixture
an organic solvent that in use is adapted to gradually evaporate, whereby said at least one resin forms a protective film that gradually releases said antibiotic mixture;
characterised in that said antibiotic mixture comprises piperacillin sodium and tazobactam sodium in a predetermined weight ratio.

2. A composition for treating peri-implantitis, according to claim 1, wherein said acrylic resin is such that said protective film has such physical characteristics that remain substantially unchanged during 7 to 10 days after which the film deteriorates and disappears from the site where it has been applied.

3. A composition for treating peri-implantitis, according to claim 1, wherein said or each biocompatible acrylic resin is selected from the group comprised of:
EUDRAGIT RL;
EUDRAGIT RS;
a mixture thereof.

4. A composition for treating peri-implantitis, according to claim 3, wherein said at least one biocompatible acrylic resin is a mixture of EUDRAGIT RL and of EUDRAGIT RS in a ratio that is set between 1, 5÷1 and 3÷1.

5. A composition for treating peri-implantitis, according to claim 1, wherein said organic solvent is ethyl alcohol.

6. A composition for treating peri-implantitis, according to claim 1, wherein said weight ratio between said tazobactam sodium and said piperacillin sodium is set between 1÷6 and 1÷10, advantageously between 1÷7 and 1÷9, preferably 1÷8.

7. A composition for treating peri-implantitis, according to claim 1, characterised in that it is topically administrated in the peri-implant region that is affected by the inflammatory process.

8. A composition for treating peri-implantitis, according to claim 1, wherein the above described composition is associated with means for administrating it to the peri-implant region close to a dental implant that is affected by the inflammatory process, in particular, a dental kit comprising a flexible blunt needle.

9. A kit for dental use, in particular, for treating peri-implantitis characterised in that it comprises:
a first vial containing a hydroalcoholic solution comprising:
an organic solvent;
at least one biocompatible acrylic resin, said or each acrylic resin being permeable, but insoluble to water;
a second vial containing a powder mixture of antibiotics, said powder mixture comprising piperacillin sodium and tazobactam sodium in a predetermined weight ratio.

10. A kit for dental use, in particular, for treating peri-implantitis, according to claim 9, wherein said weight ratio between said tazobactam sodium and said piperacillin sodium is set between 1 to 6 and 1 to 10, advantageously is set between 1 to 7 and 1 to 9, preferably is 1 to 8.

11. A kit for dental use, in particular, for treating peri-implantitis, according to claim 9, wherein said second vial contains for each 100 mg of piperacillin sodium 12,5 mg of tazobactam sodium.

12. A kit for dental use, in particular, for treating peri-implantitis, according to claim 9, wherein said second vial contains 1 ml of hydroalcoholic solution.

13. A kit for dental use, in particular, for treating peri-implantitis, according to claim 9, wherein said hydroalcoholic solution has the following composition:
EUDRAGIT RS: set between 5% and 10% by weight;
EUDRAGIT RL: set between 4% and 6% by weight;
Deionised water: set between 10% and 20% by weight;
ethyl alcohol: set between 70% and 80% by weight.

14. A kit for dental use, in particular, for treating peri-implantitis, according to claim 9, wherein at least one syringe having a perforating needle is further provided, by means of which the hydroalcoholic solution is drawn from the first vial and put in the second vial to make a resulting composition.

15. A kit for dental use, in particular, for treating peri-implantitis, according to claim 14, wherein at least one flexible blunt injection needle is furthermore provided, for administrating said resulting composition at an administration site.
